(19) United States
(12) Patent Application Publication
(10) Pub. No.: US 2012/0116252 A1
(43) Pub. Date: May 10, 2012

(54) SYSTEMS AND METHODS FOR DETECTING BODY ORIENTATION OR POSTURE

(75) Inventors: Kimberly Eileen Newman, Englewood, CO (US); Maulik Jagdishbhai Kapuria, Austin, TX (US); Niket Dhimantkumar Shah, Austin, TX (US)

(73) Assignee: THE REGENTS OF THE UNIVERSITY OF COLORADO, a body Corporate, Denver, CO (US)

(21) Appl. No.: 13/272,815

(22) Filed: Oct. 13, 2011

Related U.S. Application Data
(60) Provisional application No. 61/392,830, filed on Oct. 13, 2010.

(51) Int. Cl. A61B 5/103

(52) U.S. Cl. 600/587

(57) ABSTRACT

Systems and methods for detecting body orientation and/or posture are provided. At least one wave sensor may be configured to output waves and collect measurements data based upon the reflections of the output waves. At least one processor may be configured to receive measurements data from the at least one wave sensor and evaluate the received measurements data to determine a posture of a monitored subject. Based at least in part upon the determined posture, one or more suitable control actions may be implemented.

### CROSS-REFERENCE TO RELATED APPLICATIONS

This application claims benefit of U.S. Provisional Application No. 61/392,830 filed Oct. 13, 2010 and entitled “Low-Cost Method and Apparatus for Detecting Body Orientation,” the disclosure of which is incorporated by reference herein in its entirety.

### FIELD OF THE INVENTION

Embodiments of the invention relate generally to monitoring systems, such as security systems or healthcare monitoring systems, and more specifically to monitoring systems that are capable of detecting body orientation or posture.

### BACKGROUND OF THE INVENTION

Conventional monitoring systems, such as security monitoring systems utilized in various homes and/or businesses, typically infer the presence of subjects (e.g., human beings) by sensing motion, presence of body heat, or movement of a door or window. However, the imprecise nature of these detection methods often result in false detection or false alarms. Additionally, many security and/or healthcare scenarios that provide for well-being and peace of mind require additional information to determine if an alarm threshold is met. For example, in independent eldercare situations, it is often vital to know whether a person is walking or upright, or whether the person has fallen. Similar concerns exist with various types of animals, such as horses.

Crude methods for determining this additional information have been developed using a series of heat sensing motion detectors and monitoring for movement between rooms or by using cameras that are constantly monitored by third-party resources. The former method is often imprecise and susceptible to missing alarm instances. The latter method is typically expensive and subject to operator error due to complacency. For example, an operator may assume that a monitored person likely has not fallen because the person has not fallen for the past two years.

Accordingly, improved systems and methods for determining presence and body orientation may be desirable. Additionally, it may be desirable to detect relatively small changes in human or animal motor activity that would indicate deterioration in health and allow for intervention before an accident occurs.

### BRIEF DESCRIPTION OF THE INVENTION

Some or alt of the above needs and/or problems may be addressed by certain embodiments of the invention. Embodiments of the invention may include systems and methods for detecting body orientation or posture. According to one embodiment of the invention, there is a monitoring system configured to detect body orientation or posture. The monitoring system may include at least one wave sensor and at least one processor. The at least one processor may be configured to: receive measurements data from the at least one wave sensor; evaluate the received measurements data to determine a posture of a monitored subject; and implement, based at least in part upon the determined posture, a control action.

According to another embodiment of the invention, there is disclosed a method for detecting body orientation or posture. Measurements data may be received from at least one wave sensor by a monitoring system. The monitoring system may include one or more computers. The received measurements data may be evaluated by the monitoring system to determine a posture of a monitored subject. Based at least in part upon the determined posture, the monitoring system may direct implementation of a control action.

According to yet another embodiment of the invention, there is disclosed a method for determining body orientation or posture. Data collected by one or more wave sensors may be received by a monitoring system that includes one or more computers. The one or more wave sensors may be configured to output waves, and the received data may include information associated with respective time delays from the one or more wave sensors to a monitored subject in the path of the one or more wave sensors. The received data may be evaluated by the monitoring system to determine a position and orientation of the monitored subject. Based at least in part upon the determined position and orientation, the monitoring system may determine whether an alert action should be taken.

Additional systems, methods, apparatus, features, and aspects are realized through the techniques of various embodiments of the invention. Other embodiments and aspects of the invention are described in detail herein and are considered a part of the claimed invention. Other embodiments and aspects can be understood with reference to the description and the drawings.

### BRIEF DESCRIPTION OF THE DRAWINGS

Having thus described the invention in general terms, reference will now be made to the accompanying drawings, which are not necessarily drawn to scale, and wherein:

FIG. 1 is a schematic block diagram of one example system that may be utilized to detect body orientation or posture, according to an illustrative embodiment of the invention.

FIG. 2 is a flow diagram of an example method for monitoring a subject, according to an illustrative embodiment of the invention.

FIG. 3 is a flow diagram of an example method for monitoring a subject and determining body orientation or posture, according to an illustrative embodiment of the invention.

FIG. 4 is an example illustration of the collection of data by a plurality of wave sensors, according to an illustrative embodiment of the invention.

FIG. 5 is an example illustration of the collection of data by a wave sensor, according to an illustrative embodiment of the invention.

### DETAILED DESCRIPTION OF THE INVENTION

Illustrative embodiments of the invention now will be described more fully hereinafter with reference to the accompanying drawings, in which some, but not all embodiments of the invention are shown. Indeed, the invention may be embodied in many different forms and should not be construed as limited to the embodiments set forth herein; rather, these embodiments are provided so that this disclosure will satisfy applicable legal requirements. Like numbers refer to like elements throughout.

Disclosed are systems and methods for determining orientation and/or posture of a monitored subject. In certain embodiments, one or more wave sensors, such as ultrasonic sensors, may be utilized to monitor a desired area. A wide variety of suitable wave-emitting sensing devices may be used as desired in various embodiments of the invention. The cost and physics of sound-emitting devices provide a relatively economical solution for presence and motion detection.

In operation, a wave sensor may emit a wave, and the wave sensor may monitor reflections of the wave. For example, the time between the output of a wave and the receipt of a wave reflection may be monitored. Based upon a determined time delay from output until reflection receipt, a distance between the wave sensor and an object that caused the reflection (e.g., another surface of a monitored area, a relatively stationary object such as furniture, a monitored subject, etc.) may be determined. Additionally, in accordance with an aspect of the invention, an orientation or posture (e.g., standing, sitting, laying down, etc.) of a monitored subject may be determined. In this regard, a wide variety of enhanced monitoring services may be provided. For example, a monitoring system may determine whether a monitored subject, such as an elderly individual, has fallen down. As desired, one or more alert events may be generated, and any number of suitable control actions (e.g., alert messages, contacting authorities, etc.) may be implemented.

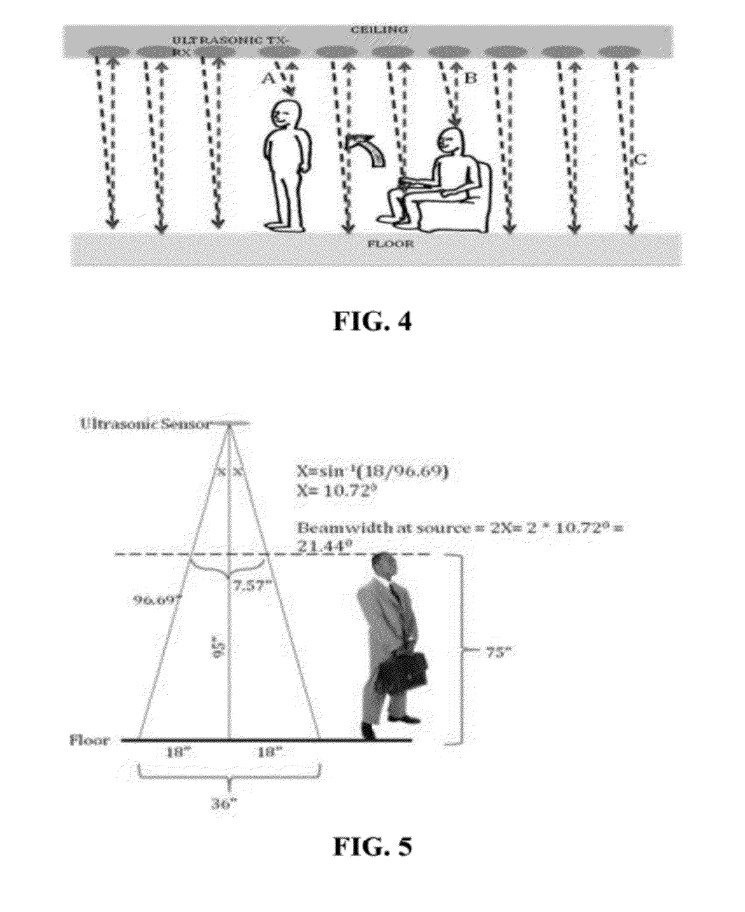

As desired in various embodiments of the invention, any number of wave-emitting sensors or wave sensors may be utilized. Additionally, a wide variety of configurations of the wave sensors may be utilized. One example configuration of sensors is described in greater detail below with reference to FIG. 4, which illustrates a plurality of wave sensors positioned on a ceiling. In other example embodiments, one or more wave sensors may be placed on any surface, such as a wall or a ceiling, or on another object associated with a monitored area. As desired, such as in a bathroom, a single sensor may be utilized. In other embodiments, a plurality of sensors may be used. For example, a plurality of sensors may be placed at predetermined intervals or positions on a ceiling. As another example, a plurality of sensors may be positioned in a suitable array or grid, and the plurality of sensors may be utilized to monitor a desired area. For example, each sensor in a grid may be positioned at a different angle in order to provide coverage for a desired area.

Additionally, as desired, sensors may be utilized to track the motion of a monitored subject. In operation, the wave nature of sound allows wave reflections to be processed in order to determine the distance between the reflecting body and the sensor. As a result, information regarding movement between grid subsections and a moving object's height may be determined. In certain embodiments, changes in orientation of the monitored subject, such as a gradual slumping of a monitored individual, may facilitate a prediction of a likely alert event (e.g., a likely fall, etc.).

In certain embodiments, the wave sensors may communicate with one or more suitable monitoring system control units, such as one or more local processing units and/or one or more remote processing units (e.g., a central monitoring server, etc.). In one example operation, the local processing unit(s) may capture information from the sensors and/or may receive measurements data (e.g., timing information, etc.) from the sensors. The local processing unit(s) may then evaluate the collected data in order to determine body orientation and/or posture associated with a monitored subject. The orientation and/or posture may then be analyzed in order to determine whether any alerts and/or other control actions should be triggered. In certain embodiments, a local processing unit may communicate with a central monitoring server or other remote processing unit. As desired, a local processing unit may complete intermediate processing of the information collected from the sensor(s) and may send the information to a central server. The central server may complete data processing and may return control signals back to the local processing unit.

As desired, the local processing unit may be a gateway device, a local computer, a camera or any other device with adequate processing capability and network connectivity. The local processing unit may coordinate the provision of control information between the sensors and other devices. For example, if sensors are deployed that utilize wireless communication and a battery power source, it may be desirable to operate the sensors in a keep-alive mode to conserve power and prolong battery life. The wave sensors may be activated by sensing gross activity or presence of a subject to be monitored. For example, by using a device such as a body heat motion detector or another convenient device/method, the wave sensors may be activated once a subject enters a monitoring area.

A wide variety of suitable algorithms and/or other processes may be utilized to evaluate data received from wave sensors. In certain embodiments, stored profile information, such as stored information associated with an actual height or range of expected heights for a standing or seated individual, may be accessed. At least a portion of the accessed profile information may then be compared to the data received from the wave sensors in order to determine a body orientation or posture of a monitored subject. Additionally, as desired in various embodiments, stored profile information associated with relatively stationary objects within a monitored area, such as furniture, may be accessed. In this regard, received measurements data indicating these objects may be filtered out from an orientation analysis. Additionally, a determination may be made as to whether a monitored subject is interacting with an object. For example, a determination may be made as to whether a monitored individual is sitting in a chair.

Various embodiments of the invention may include one or more special purpose computers, systems, and/or particular machines that facilitate the determination of body orientation and/or posture. A special purpose computer or particular machine may include a wide variety of different software modules as desired in various embodiments. As explained in greater detail below, in certain embodiments, these various software components may be utilized to detect or determine body orientation and/or posture and to trigger a wide variety of suitable alerts and/or other actions based upon the body orientation and/or posture.

Structural Overview

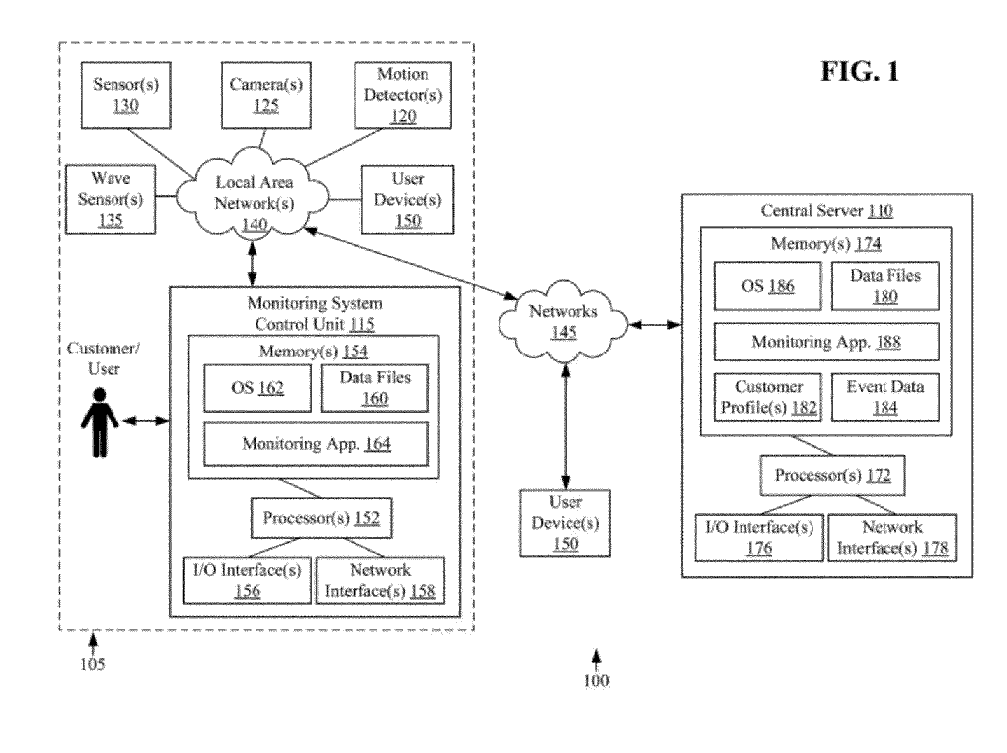

FIG. 1 illustrates one example system 100 that may be utilized to detect or determine body orientation and/or posture. With reference to FIG. 1, the system 100 may include a wide variety of components that are situated within or within relatively close proximity to a structure that is monitored, such as a home, a business, a stable, or other structure. For example, various system components may be situated within a household 105. Additionally, the system 100 may include a central server 110 configured to receive data, such as sensor data and/or monitoring data from devices associated with the household 105.

For purposes of this disclosure, the entire system 100 may be referred to as a “monitoring system” with the components associated with the household 105 being referred to as a “local monitoring system.” However, for simplicity, the components associated with the household 105 may be referred to as a monitoring system rather than a local monitoring system. Such language should not be construed as limiting the meaning of the term “monitoring system” to local components associated with household 105.

With reference to the household 105, a monitoring system control unit 115 and/or any number of sensing devices, such as motion detectors 120, cameras 125, and/or other sensors 130 (e.g., microphones or voice detectors, smoke detectors, contact sensors, etc.) may be provided in association with any number of wave sensors 135. As desired, the control unit 115 may communicate with the various sensors via any number of local networks 140 or household networks, such as a local area network, a home area network (“HAN”), a Bluetooth-enabled network, a Wi-Fi network, a wireless network, a suitable wired network, etc. As desired, the control unit 115 may additionally communicate with any number of user devices 150 via the local networks 140, such as a mobile device or other device associated with a user.

Additionally, the control unit 115 and/or any number of the sensors 120, 125, 130, 135 may communicate with any number of external devices, such as the central server 110, via any number of suitable external networks 145, such as a cellular network, a public-switched telephone network, an Advanced Metering Infrastructure (“AMI”) network, the Internet, and/or any other suitable public or private network. As desired, the user devices 150 may also communicate with the central server 110 and/or the monitoring system control unit 115 via the external networks 145.

In certain embodiments, the control unit 115 may be a standalone device, such as a monitoring system panel that includes suitable hardware and/or software components. In other embodiments, the control unit 115 may be integrated into one or more of the other illustrated system components 120, 125, 130, 135. In yet other embodiments, the control unit 115 may be integrated into a wide variety of other devices not illustrated in FIG. 1, such as a utility meter or a home power management system. As desired, the functionality of the control unit 115 may also be distributed among a plurality of different devices.

The control unit 115 may be a suitable processor-driven device that facilitates the management of a monitoring system, such as a household monitoring system. Additionally, in certain embodiments, the control unit 115 may be a suitable processor-driven device that facilitates the evaluation of parameters and/or monitoring data in order to determine a position, orientation, and/or posture associated with one or more monitored subjects. Examples of suitable devices that may be utilized for and/or associated with the control unit 115 include, but are not limited to, personal computers, microcontrollers, minicomputers, and/or other suitable processor-driven devices. The one or more processors 152 associated with the control unit 115 may be configured to execute computer-readable instructions in order to form a special purpose computer or particular machine that is configured to manage a local monitoring system and/or to facilitate the determination of position, orientation, and/or posture associated with one or more monitored subjects.

In addition to having one or more processors 152, the control unit 115 may include one or more memory devices 154, one or more input/output (“I/O”) interfaces 156, and/or one or more network interfaces 158. The memory devices 154 may include any suitable memory devices and/or data storage elements, such as read-only memory devices, random access memory devices, magnetic storage devices, etc. The memory devices 154 may be configured to store a wide variety, of information, for example, data files 160, user profile data, and/or any number of software modules and/or executable instructions that may be executed by the one or more processors 152, such as an operating system (“OS”) 162, and/or a monitoring application 164.

The data files 160 may include any suitable data that facilitates the operation of the control unit 115, such as data that facilitates identification of the one or more sensors 120, 125, 130, 135, data that facilitates communication with the sensors 120, 125, 130, 135, data that facilitates identification of and/or communication of the user devices 150, data that facilitates communication with the central server 110, collected monitoring data, user profile data, and/or profile data associated with one or more monitored areas. The OS 162 may be a suitable software module that facilitates the general operation of the control unit 115. Additionally, the OS 162 may facilitate the execution of any number of other software modules, such as the monitoring application 164.

In operation, the control unit 115 may facilitate the management of a local monitoring system, such as a household monitoring system. For example, the control unite 115 may communicate with one or more sensors 120, 125, 130, 135 and/or user devices 150 in order to determine when an alarm event or other event should be triggered. For example, an alarm event may be triggered by a security monitoring system based upon the identification of a break-in or unauthorized entry to a monitored area. As desired, a monitoring application associated with the control unit 115 and/or a central server 110 in communication with the control unit 115 may facilitate the triggering of alarm events.

As desired, a monitoring application 164 associated with the control unit 115 and/or a central server 110 in communication with the control unit 115 may facilitate the collection of monitoring data, the identification of alarm events, and/or the execution of one or more control actions based upon triggered alarm events. The monitoring application 164 may be a suitable software module that receives the various inputs from sensors 120, 125, 130, 135 and executes one or more action(s) based at least in part upon detected alarm events (e.g., break-in events, body orientation events, etc.) and/or instructions received from the central server 110.

A wide variety of suitable operations may be performed by the monitoring application 164 as desired in various embodiments of the invention. For example, the monitoring application may identify one or more sensors associated with a monitored area. These sensors may include one or more wave sensors 135. Additionally, the monitoring application 164 may determine a wide variety of profile information associated with the sensors (e.g., a covered area, configuration data etc.), the monitored area (e.g., positions and/or dimensions of relatively stationary objects, etc.), and/or one or more subjects to be monitored (e.g., a standing height of an individual, a sitting height of an individual, a range of expected values associated with sitting and/or standing heights, etc.). In certain embodiments, at least a portion of the profile information may be collected during a learning mode and/or configuration mode of the monitoring application 164. For example, wave sensors may be utilized to determine one or more heights or other dimensions associated with a subject to be monitored, and dimension information may be stored. As another example, wave sensors may be utilized to determine dimensions of one or more objects in a monitored area, and at least a portion of the dimension information (as well as position information) may be stored.

In certain embodiments, the monitoring application 164 may activate one or more wave sensors 135 based upon a detected presence of a subject to be monitored. For example, data collected from a suitable motion detector 120 may be evaluated in order to determine the presence of a subject, and the wave sensors 135 may be activated by the monitoring application 164 based at least in part upon the detected presence. Once activated, the wave sensors 135 may take measurements of the monitored area (e.g., timing measurements for wave reflections, etc.), and measurements data may be received and processed by the monitoring application 164. In this regard, the monitoring application 164 may track a subject located within the monitored area. Additionally, according to an aspect of the invention, the monitoring application 164 may determine an orientation or posture associated with the monitored subject. For example, the monitoring application 164 may evaluate measurements data to determine a height or other dimensions associated with the monitored subject. A determined dimension may then be compared to one or more stored or expected values (or ranges of values and/or threshold values). Based at least in part upon the comparison, an orientation or posture of the monitored subject may be determined. For example, a determination may be made as to whether a monitored subject is standing, sitting, or laying down.

Once an orientation or posture has been determined, a determination may be made as to whether an alert event should be triggered or generated by the monitoring application 164. For example, a determination may be made as to whether a monitored subject has fallen down. As another example, a determination may be made as to whether a monitored subject is slumping and likely to fall down. Once an alert event has been triggered, the monitoring application 164 may direct a wide variety of suitable control actions based at least in part upon the triggered alert event. Examples of suitable control actions include, but are not limited to the activation of one or more additional sensors and/or monitoring devices (e.g., cameras 125, audio receivers, etc.) that facilitate additional monitoring (e.g., monitoring by security personnel, etc.), the communication of an alert message (e.g., communicating a message to emergency personnel, communicating a message to a relative or other individual, communicating a message to monitoring system personnel, etc.), the communication of a message (e.g., a status message to determine whether a monitored subject needs help, etc.) to a device (e.g., a user device 150) associated with the monitored subject, and/or the escalation of an alert that has not been closed.

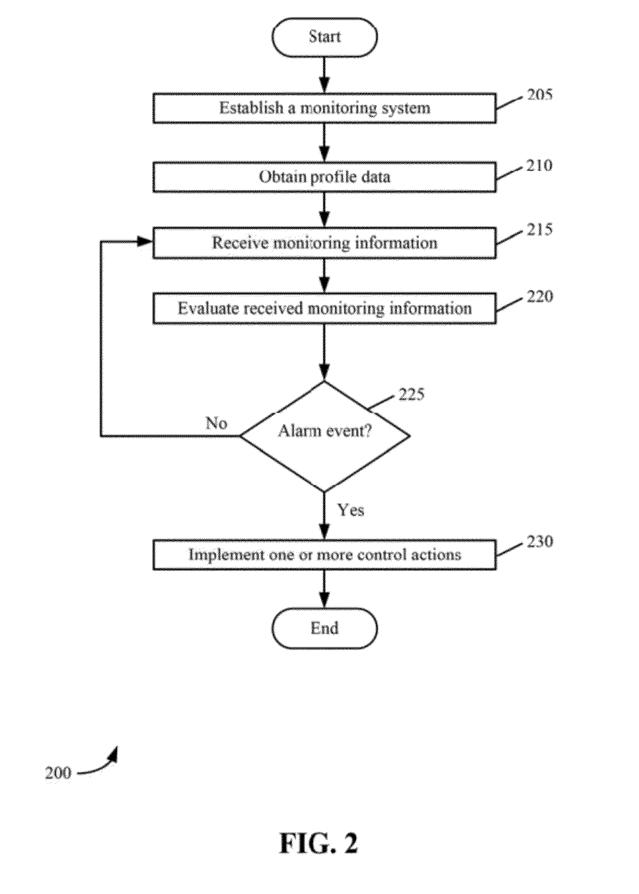

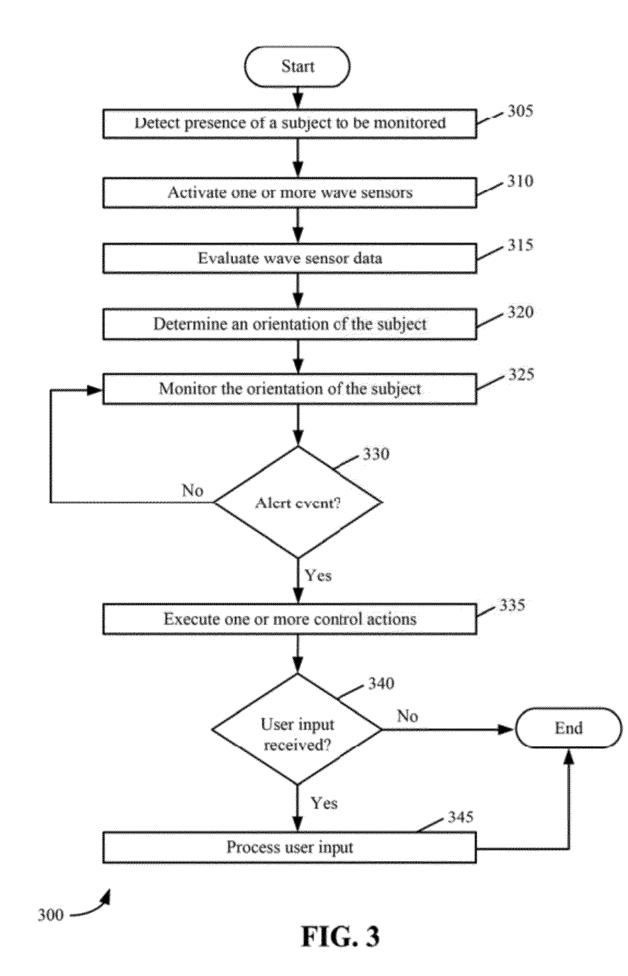

A few examples of the operations that may be performed by the monitoring application 164 are described in greater detail below with reference to FIGS. 2 and 3.

With continued reference to the control unit 115, one or more input/output (“I/O”) interfaces 156 may facilitate interaction with any number of I/O devices that facilitate the receipt of user and/or device input by the control unit 115, such as a keyboard, a touch screen display, a microphone, etc. Additionally, the one or more network interfaces 158 may facilitate connection of the control unit 115 to any number of suitable networks, such as the local area networks 140 and/or the external networks 145. In this regard, the control unit 115 may communicate with any number of other components of the system 100. For example, the control unit 115 may receive data from sensors 120, 125, 130, 135 and/or user devices 150. As another example, the control unit 115 may communicate commands to the various sensors 120, 125, 130, 135. As yet another example, the control unit 115 may communicate data to and/or receive data from the central server 110.

With continued reference to FIG. 1, the central server 110 may be a suitable processor-driven device configured to receive data from any number of local control units 115 and/or to determine a wide variety of actions based upon an evaluation of the received data. For example, in certain embodiments, the central server 110 may evaluate wave sensor data in order to determine orientation and/or posture. As another example, the central server 110 may receive alert and/or other event information, and the central server 110 may process the received alert information. For example, the central server 110 may direct monitoring personnel to view a camera feed of the monitored area in order to determine whether a monitored subject has fallen. Examples of suitable processor-driven devices that may be utilized for the central server may include any number of suitable server computers, personal computers, minicomputers, microcontrollers, and/or other processor-based devices. In certain embodiments, the central server 110 may execute computer-executable instructions that form a special purpose computer or particular machine that facilitates the determination of whether an associated monitoring system has detected an event that should trigger an alert. Although the central server 110 is described in greater detail below, at least a portion of the operations of the central server 110 described below and/or at least a portion of the operations described with reference to FIGS. 2 and 3 may be performed by the monitoring system control unit 115.

In addition to having one or more processors 172, the central server 110 may include any one or more suitable memory devices 174, one or more suitable input/output (“I/O”) interfaces 176, and/or one or more suitable network interfaces 178. The memory devices 174 may include any suitable memory devices, such as read-only memory devices, random access memory devices, magnetic storage devices, etc. The memory devices 174 may be configured to store a wide variety of data utilized by the central server 110, for example, data files 180, one or more customer profile databases 182, one or more event data'databases 184, and/or any number, of other databases and/or other logical memory constructs. Additionally, the memory devices 174 may be configured to store various software modules and/or executable instructions that may be executed by the one or more processors 172, such as a monitoring application 188.

The data files 180 may include any suitable data that facilitates the general operation of a central server 110, the determination of position, orientation, and/or posture, and/or the processing of received alert and/or event data. For example, the data files 180 may include various settings information associated with any number of household monitoring systems. As another example, the data files 180 may include contact information and/or network data associated with the household monitoring systems. As other examples, the data files 180 may include received measurements data (e.g., data collected by the sensors 120, 125, 130, 135) and/or received data associated with determined orientations, positions, and/or postures. The customer profile database 182 may include, for example, various application rules, preferences, and/or user profiles associated with one or more customers and/or one or more subjects to be monitored, such as profile information utilized in orientation and/or posture determinations (e.g., dimensions of subjects to be monitored, dimensions associated with relatively stable objects, etc.) and/or profile information associated with desired control actions to take based upon identified alerts. The event data database 184 may include, for example, data associated with identified events, such as information associated with generated alert events and/or information associated with received alert events. A wide variety of different files and/or logical memory constructs may be utilized to store data that is utilized in various embodiments of the invention. The various files and databases that are described above are provided by way of example only and should not be construed as limiting.

The operating system (“OS”) 186 may be a suitable software module that facilitates the general operation of the central server 110. Additionally, the OS 186 may facilitate the execution of any number of other software modules and/or applications, such as the monitoring application 188. The monitoring application 188 may be a suitable software module that receives various inputs and/or alerts from sensors, user devices, etc., and executes one or more action(s) based upon processing the received information. For example, the monitoring application 188 may identify alarm events, and trigger an alarm and/or other control actions (e.g., escalation of an alarm, contacting a customer, etc.) in association with the identification of an alarm event. As another example, in certain embodiments, the monitoring application 188 may determine position, orientation, and/or posture information associated with a monitored subject, and the monitoring application 188 may determine whether one or more control actions should be taken. Indeed, in certain embodiments, operations performed by the monitoring application 188 may be similar, to those described above for the monitoring application 164 of the control unit 115. However, as desired, other operations may be performed. For example, the monitoring application 188 may direct monitoring personnel to review a camera feed or to attempt to establish contact with a monitored subject once an alert event has been identified.

A few examples of the operations that may be performed by the monitoring application 188 are described in greater detail below with reference to FIGS. 2 and 3.

With continued reference to the central server 110, one or more input/output (“I/O”) interfaces 176 may facilitate interaction with any number of I/O devices that facilitate the receipt of user and/or device input by the central server 110, such as a keyboard, a mouse, a touch screen display, a microphone, etc. Additionally, the one or more network interfaces 178 may facilitate connection of the central server 110 to any number of suitable networks, such as a cellular network, a public-switched telephone network, the Internet, etc., that facilitate communications between the central server 110 and one or more other components of the system 100, such as the monitoring system control unit 115 and/or any number of user devices 150, such as a mobile device of a user. In this regard, the central server 110 may receive monitoring and/or, measurements data from the control unit 115. Additionally, as desired, the central server 110 may receive user commands and/or requests for data from the control unit 115 and/or the user devices 150.

With, continued reference to FIG. 1, any number of user devices 150 may be provided. One example of a suitable user device 150 is a mobile device (e.g., a mobile telephone, a personal digital assistant, etc.), although other types of user devices may be utilized, such as tablet computers, digital readers, etc. In certain embodiments, a user may utilize a user device 150 to provide commands to and/or receive data from one or more other components of the system 100. For example, a user device 150 may be configured to receive alarm data and/or event data from the control unit 115 and/or the central server 110, and at least a portion of the received data may be presented to a user. As another example, a user may utilize a user device 150 to provide any number of commands associated with the monitoring system to the control unit 115 and/or the central server 110, such as a request to escalate, an alert or an indication that an alert is associated with a false alarm.

As desired, embodiments of the invention may include a system 100 with more or less than the components illustrated in FIG. 1. The system 100 of FIG. 1 is provided by way of example only.

Operational Overview

FIG. 2 is a flow diagram of an example method 200 for monitoring a subject, according to an illustrative embodiment of the invention. Various operations of the method 200 may be performed by a monitoring system control unit and/or by a central server, such as the control unit 115 and/or central server 110 illustrated in FIG. 1. For example, various operations of the method 200 may be performed by a suitable monitoring application associated with the control unit 115 and/or by a suitable monitoring application associated with the central server 110, such as one or both of the monitoring applications 164, 188 illustrated in FIG. 1. The method may begin at block 205.

At block 205, a monitoring system may be installed, established, and/or initiated. As desired, the monitoring system may be configured to conduct a wide variety of different types of monitoring, such as security monitoring and/or healthcare monitoring. According to an aspect of the invention, the monitoring system may include any number of suitable wave sensors, such as ultrasonic sensors. The wave sensors may be positioned in a wide variety of suitable configurations (e.g., a plurality of wave sensors spaced along a ceiling, a group of wave sensors placed in a corner of a room, etc) in order to cover a desired monitoring area. In this regard, the wave sensors may monitor one or more subjects (e.g., individuals, animals, etc.) positioned within the monitoring area to facilitate a determination of position, orientation, and/or posture.

At block 210, a wide variety of profile data may be obtained. One example of suitable profile data includes data associated with one or more subjects to be monitored. For example, dimensional information associated with one or more subjects may be obtained for a wide variety of different orientations and/or postures (e.g., standing, sitting, laying down, etc). As another example, default profile information, such as one or more threshold values and/or ranges of values associated with the likely orientation and/or postures of subjects to be monitored. As yet another example, profile information associated with one or more relatively stationary objects situated within a monitored area may be obtained. For example, dimensional information associated with furniture and/or other objects positioned within a monitored room may be obtained.

As desired, profile data may be obtained utilizing a wide variety of suitable techniques. For example, a user of the monitoring system may utilize one or more suitable input devices to provide profile data to the monitoring system. As another example, profile information may be entered via any number of suitable Web pages and/or graphical user interfaces hosted by a suitable server (e.g., a Web server, etc) associated with the monitoring system. As yet another example, the monitoring system may be placed in a configuration and/or learning mode. While in the learning mode, the one or more wave sensors may take one or more measurements utilized to generate steady-state or baseline information (e.g., dimensions, etc.) associated with the monitored area and/or one or more subjects to be monitored.

At block 215, the monitoring system may be activated, and monitoring information may be collected. For example, measurements data (e.g., motion detection indications, timing information, etc.) and/or other monitoring information may be generated and/or collected by any number of suitable sensors, and at least a portion of the collected data may be provided to a suitable processing system or device. At block 220, the processing system or device may evaluate and/or analyze the received monitoring information. In this regard, a wide variety of suitable events may be identified and/or any number of suitable determinations may be made. For example, wave sensor measurements may be utilized in association with profile information in order to determine an orientation or posture associated with a monitored subject. The orientation or posture may then be evaluated in order to determine whether an alarm event or an alert should be triggered.

At block 225, a determination may be made as to whether any alarm events or alert events have been detected. For example, a determination may be made as to whether a determined orientation or posture indicates that a monitored subject has fallen. As another example, a determination may be made as to whether a monitored subject's posture has degraded (e.g., a monitored subject is slumping) in a manner indicating that the monitored subject may likely fall or otherwise experience a health problem. In security monitoring systems, another example determination may determine whether an unauthorized entry has been made. If it is determined at block 225 that an alarm event has not been detected, then operations may continue at block 215, and monitoring may continue. If, however, it is determined at block 225 that an alarm event has been detected, then operations may continue at block 230.

At block 230, one or more suitable control actions may be implemented based at least in part upon the determined or identified alarm event. As one example control action, one or more suitable alarm or alert messages may be communicated to one or more designated recipients, such as a monitoring center, an emergency responder, or a suitable user device. A wide variety of suitable communication techniques and/or communication channels may be utilized to communicate an alert message, such as email, a telephone call, or a short message service (“SMS”) message. As another example control action, an attempt to establish contact with the monitored subject may be made in order to determine whether assistance is needed. For example, an attempt may be made to establish contact via a suitable user device associated with the monitored person, such as a mobile device. As another example, an attempt may be made to establish contact via a bi-directional communication channel associated with the monitoring system.

As yet another example control action, additional monitoring may be directed. For example, one or more cameras and/or audio receivers or detection devices (e.g., microphones, other audio receivers, etc) may be activated. As desired, data collected by the activated devices may be provided to suitable monitoring personnel, such as security or healthcare personnel. In this regard, a determination may be made as to whether an alarm event should be escalated. For example, a camera feed may be evaluated by monitoring personnel in order to verify that a monitored subject has fallen, and the monitoring personnel may escalate the alert and direct an emergency response.

The method 200 may end following block 230.

FIG. 3 is a flow diagram of an example method 300 for monitoring a subject and determining body orientation or posture, according to an illustrative embodiment of the invention. In certain embodiments, the operations of the method 300 are one example of the operations that may be performed at blocks 215-230 illustrated in FIG. 2. As desired, various operations of the method 300 may be performed by a monitoring system control unit and/or by a central server, such as the control unit 115 and/or central server 110 illustrated in FIG. 1. For example, various operations of the method 300 may be performed by a suitable monitoring application associated with the control unit 115 and/or by a suitable monitoring application associated with the central server 110, such as one or both of the monitoring applications 164, 188 illustrated in FIG. 1. The method 300 may begin at block 305.

At block 305, the presence of a subject to be monitored may be detected. As desired, presence may be detected by any number of suitable detection devices associated with a monitored area of interest. For example, one or more suitable motion detectors may be utilized to detect presence of an individual entering a monitored area and/or located within the monitored area. A motion detector 120 may be, for example, a traditional body heat sensor, a camera, a door contact, etc.

At block 310, a monitoring system (e.g., a control unit 115, etc.) may receive measurements data and/or a presence detection indication from the detection devices. In this regard, the monitoring system may determine that a subject to be monitored is located within an area of interest. Based at least in part upon the detected presence of a subject to be monitored, one or more suitable wave sensors associated with the area, such as the wave sensors 135 illustrated in FIG. 1, may be activated. For example, a control unit 115 may send one or more suitable signals to the wave sensors 135 in order to awaken and/or activate the wave sensors 135.

At block 315, the wave sensors 135 may output sound waves and receive reflection data associated with the output waves. The wave sensors 135 may then communicate the reflectance distance data and/or timing data associated with the detected reflections to the control unit 115. The control unit 115, either alone or in conjunction with a central server 110, may receive the measurements data. The received measurements data may be processed and/or evaluated utilizing a wide variety of suitable evaluation techniques. For example, the measurements data may be compared to baseline data and/or expected data, such as data stored in a suitable profile. In this regard, deviations from the baseline data may be determined.

At block 320, orientation, position, and or posture of a monitored subject may be determined. For example, differences between baseline data (or expected data) and monitored data may be identified in order to determine a position of a monitored subject situated within the sound wave sensor 135 grid. Additionally, the position may be evaluated utilizing a wide variety of suitable threshold values and/or ranges of values in order to determine an orientation or posture of the monitored subject. For example, a determination may be made as to whether the monitored subject is standing, sitting, or in a prone or laying down position. As desired, profile information associated with one or more relatively stable objects (e.g., furniture) may also be taken into consideration when determining a position, orientation, and/or posture of a monitored subject. For example, in certain embodiments, the profile information may be utilized to establish baseline data. In other embodiments, the profile information may be utilized in association with, an identified position of the monitored subject to determine a posture of the monitored subject. For example, if the monitored subject's position is the same as a chair or a bed, then a determination may be from evaluating the measurements data that the monitored subject is sitting in the chair or laying on the bed.

At block 325, the orientation or posture of the subject may be periodically or continually monitored while the subject is located within the monitored area. For example, the control unit 115 and/or central server 110 may monitor measurements data in an attempt to identify changes in orientation and movement within the wave sensor 135 grid. As one example, sensor 135 data may be periodically received, analyzed, and/or compared to previous data in order to identify changes in movement and/or position.

At block 330, a determination may be made as to whether any alert events or alarm events are identified. For example, a determination may be made as to whether a determined orientation or posture (or change in orientation or posture) indicates that a monitored subject has fallen. As another example, a determination may be made as to whether a monitored subject's posture has degraded (e.g., a monitored subject is slumping) in a manner indicating that the monitored subject may likely fall or otherwise experience a health problem. If it is determined at block 330 that an alert event has not been detected, then operations may continue at block 325, and monitoring may continue. If, however, it is determined at block 330 that an alert event has been detected, then operations may continue at block 335.

At block 335, any number of suitable control actions may be executed, triggered, and/or directed based at least in part upon the identification of the alert event. An example of a control action may include the communication of a notification to a user(s), such as an email notification, short message service notification, and/or telephone call. For example, if it is determined that an elderly person has likely fallen, then an alert message may be communicated to a relative and/or to emergency personnel. As another example, additional monitoring may be implemented, such as additional monitoring by a camera or an audio receiver. Alternatively, in certain embodiments, no action may be triggered. For example, if the wave sensor measurements data indicates that a monitored subject has left, a monitored area (e.g., a room, etc.), then no action may be triggered or wave sensors situated in an area that the monitored subject entered may be activated.

At block 340, a determination may be made as to whether user input associated with an executed control action has been received. A wide variety of suitable user input may be received in various embodiments of the invention. For example, user input instructing the monitoring system 100 to activate one or more cameras 125, deactivate the system, and/or update user preferences and/or user profile information may be received. As another example, user input may indicate that a monitored subject is okay and that an alarm condition should be closed. As another example, user input may additionally be utilized in an evaluation of whether a control action or alarm should be escalated. For example, if it is determined based upon an analysis of saved sensor data that a person has likely fallen, then a camera may be activated and monitoring system personnel may observe the area in which the person is believed to have fallen. If the monitoring system personnel conclude that the person has fallen, then emergency personnel may be contacted.

If it is determined at block 340 that user input has been received, then operations may continue at block 345, and the received user input may be processed. In this regard, a wide variety of additional control actions (e.g., additional monitoring, alarm escalation, closing an alarm, etc.) may be implemented and/or executed. Operations may then end following block 345. If, however, it is determined at block 340 that no user input has been received, then operations may end.

The method 300 may end following either block 340 or block 345. Alternatively, operations may continue at block 315, and monitoring may continue.

The operations described in the methods 200, 300 of FIGS. 2 and 3 do not necessarily have to be performed in the order set forth in FIGS. 2 and 3, but instead may be performed in any suitable order. Additionally, in certain embodiments of the invention, more or less than all of the elements or operations set forth in FIGS. 2 and 3 may be performed.

FIG. 4 is an example illustration of the collection of data by a plurality of wave sensors, according to an illustrative embodiment of the invention. With reference to FIG. 4, a plurality of wave sensors (e.g., ultrasonic sensors) are positioned along the surface of a ceiling. For example, wave sensors are positioned in a pattern and/or in accordance with predetermined intervals. Each of the wave sensors may be configured to output one or more waves (e.g., acoustic waves, etc.) and detect reflections of the waves. A wave sensor may additionally be configured to measure or determine a timing delay from the output of a wave until a reflection is detected. In this regard, a distance between the wave sensor and a reflective surface (e.g., the floor, a chair, etc) or subject (e.g., a monitored subject, etc.) may be determined. Based at least in part upon these measured distances, an orientation or posture of a monitored subject may be, determined.

For example, with reference to FIG. 4, a first measured distance “A” may be determined when a monitored subject is situated in a standing position below a wave sensor. Based at least in part upon the first measured distance, a determination may be made that the monitored subject is standing. As another example, when the monitored subject is in a sitting position, a second measured distance “B” (e.g., a distance between the subject's head and the sensor, etc.) may be determined. As desired, other measured distances, such as a distance between a sensor and the subject's lap, may be determined. Based at least in part upon these one or more measurements (and optionally on profile information identifying a position of a chair), a determination may be made that the monitored subject is in a seated position. In the event that the subject falls down, the measured distances may be processed in order to determine that the subject is in a prone or laying down position, and a suitable alarm event may be generated.

The configuration of wave sensors illustrated in FIG. 4 is provided by way of example only. It will be appreciated that a wide variety of other wave sensor configurations may be utilized as desired in various embodiments of the invention. For example, a group or array of wave sensors may be positioned in a corner of the ceiling and utilized to monitor a desired area.

FIG. 5 is an example illustration of the collection of data by a wave sensor, according to an illustrative embodiment of the invention. The wave sensor may be configured to emit, or output an acoustic signal. The acoustic signal may propagate outwardly from the wave sensor. For example, the acoustic signal may propagate outwardly in a direction perpendicular to a surface to which the wave sensor is attached. The acoustic signal may also propagate outwardly at any number of other angles. Additionally, the acoustic signal may expand or dissipate the greater the distance from the wave sensor.

The acoustic signal may travel away from the wave sensor until it encounters an object or surface that reflects the acoustic signal. For example, the acoustic signal may reflect off of a surface opposite to the surface to which the wave sensor is attached. As another example, the acoustic signal may reflect off of a monitored subject. At least a portion of the reflected signal may be detected by the wave sensor as it is returned to the wave sensor. As desired, a time delay from the output of the signal until the detection of a reflection may be detected or determined. In this regard, a distance between the wave sensor and the reflective surface of an object may be determined. For a monitored subject, an orientation or posture may also be determined.

As desired, a wave sensor may be deployed at a wide variety of different heights. As shown in FIG. 5, a wave sensor may be deployed at a height of approximately ninety-five inches (95″). A beam or signal emitted by the wave sensor may have an associated beam width defining a coverage area for the wave sensors. For the sensor illustrated in FIG. 5, the beam or signal may cover an area of approximately thirty-six inches (36″). For example, the beam may cover approximately 36″ of floor space underneath the sensor. As shown, the 36″ of coverage may extend approximately 18″ from a central point positioned underneath the wave sensor. Accordingly, a beam width for the sensor may be calculated as approximately 21.44 degrees. The beam width may be calculated as twice the inverse of the sine of the projection of the beam on the floor divided by the hypotenuse of the illustrated triangle. The illustrated beam width is provided by way, of example only, and other beam widths may be utilized as desired in various embodiments of the invention.

The invention is described above with reference to block and flow diagrams of systems, methods, apparatuses, and/or computer program products according to example embodiments of the invention. It will be understood that one or more blocks of the block diagrams and flow diagrams, and combinations of blocks in the block diagrams and flow diagrams, respectively, can be implemented by computer-executable program instructions. Likewise, some blocks of the block diagrams and flow diagrams may not necessarily need to be performed in the order presented, or may not necessarily need to be performed at all, according to some embodiments of the invention.

These computer-executable program instructions may be loaded onto a general purpose computer, a special-purpose computer, a processor, or other programmable data processing apparatus to produce a particular machine, such that the instructions that execute on the computer, processor, or other programmable data processing apparatus create means for implementing one or more functions specified in the flow diagram block or blocks. These computer program instructions may also be stored in a computer-readable memory that can direct a computer or other programmable data processing apparatus to function in a particular manner, such that the instructions stored in the computer-readable memory produce an article of manufacture including instruction means that implement one or more functions specified in the flow diagram block or blocks. As an example, embodiments of the invention may provide for a computer program product, comprising a computer usable medium having a computer-readable program code or program instructions embodied therein, said computer-readable program code adapted to be executed to implement one or more functions specified in the flow diagram block or blocks. The computer program instructions may also be loaded onto a computer or other programmable data processing apparatus to cause a series of operational elements or steps to be performed on the computer or other programmable apparatus to produce a computer-implemented process such that the instructions that execute on the computer or other programmable apparatus provide elements or steps for implementing the functions specified in the flow diagram block or blocks.

Accordingly, blocks of the block diagrams and flow diagrams support combinations of means for performing the specified functions, combinations of elements or steps for performing the specified functions and program instruction means for performing the specified functions. It will also be understood that each block of the block diagrams and flow diagrams, and combinations of blocks in the block diagrams and flow diagrams, can be implemented by special-purpose, hardware-based computer systems that perform the specified functions, elements or steps, or combinations of special purpose hardware and computer instructions.

While the invention has been described in connection with what is presently considered, to be the most practical and various embodiments, it is to be understood that the invention is not to be limited to the disclosed embodiments, but on the contrary, is intended to cover various modifications and equivalent arrangements included within the scope of the appended claims.

This written description uses examples to disclose the invention, including the best mode, and also to enable any person skilled in the art to practice the invention, including making and using any devices or systems and performing any incorporated methods. The patentable scope of the invention is defined in the claims, and may include other examples, that occur to those skilled in the art. Such other examples are intended to be within the scope of the claims if they have structural elements that do not differ from the literal language of the claims, or if they include equivalent structural elements with insubstantial differences from the literal language of the claims.

1. A monitoring system comprising:
at least one wave sensor; and
at least one processor configured to:
receive measurements data from the at least one wave sensor;
evaluate the received measurements data to determine a posture of a monitored subject; and
implement, based at least in part upon the determined posture, a control action.

2. The monitoring system of claim 1, wherein the at least one wave sensor comprises at least one ultrasonic sensor.

3. The monitoring system of claim 1, wherein the at least one wave sensor is mounted to at least one of (i) a wall or (ii) a ceiling.

4. The monitoring system of claim 1, wherein the at least one wave sensor comprises a plurality of wave sensors.

5. The monitoring system of claim 1, further comprising:
at least one sensor configured to detect presence of the monitored subject,
wherein the at least one wave sensor is activated based at least in part upon the detected presence.

6. The monitoring system of claim 1, wherein the at least one processor is further configured to:
identify profile information associated with the monitored subject;
compare the received measurements data to the profile information; and
determine the posture of the monitored subject based at least in part upon the comparison.

7. The monitoring system of claim 1, wherein the at least one processor is further configured to:
identify profile information for a monitored area associated with the at least one wave sensor; and
determine the posture of the monitored subject based at least in part upon the profile information to.

8. The monitoring system of claim 1, wherein the control action comprises one of (i) triggering an alarm or (ii) communicating an alert message.

9. The monitoring system of claim 1, wherein the at least one processor is further configured to:
direct, based at least in part upon the determined posture, activation of one or more cameras and/or audio receivers to facilitate additional monitoring of the monitored subject.

10. The monitoring system of claim 1, wherein the at least one processor comprises a plurality of processors with a first processor positioned at a location associated with the monitored subject and a second processor positioned at a remote processing location.

11. A method comprising:
receiving, by a monitoring system comprising one or more computers, measurements data from at least one wave sensor;
evaluating, by the monitoring system, the received measurements data to determine a posture of a monitored subject; and
directing, by the monitoring system based at least in part upon the determined posture, implementation of a control action.

12. The method of claim 11, wherein receiving measurements data from at least one wave sensor comprises receiving measurements data from at least one ultrasonic sensor.

13. The method of claim 11, wherein receiving measurements data from at least one wave sensor comprises receiving measurements data from at least one wave sensor mounted to at least one of (i) a wall or (ii) a ceiling.

14. The method of claim 11, wherein receiving measurements data from at least one wave sensor comprises receiving measurements data from a plurality of wave sensors.

15. The method of claim 11, further comprising:
detecting, by the monitoring system, presence of the monitored subject; and
directing, by the monitoring system, activation of the at least one wave sensor based at least in part upon the detected presence.

16. The method of claim 11, further comprising:
identifying, by the monitoring system, profile information associated with the monitored subject;
comparing, by the monitoring system, the received measurements data to the profile information; and
determining, by the monitoring system, the posture of the monitored subject based at least in part upon the comparison.

17. The method of claim 11, further comprising:
identifying, by the monitoring system, profile information for a monitored area associated with the at least one wave sensor; and
determine the posture of the monitored subject based at least in part upon the profile information.

18. The method of claim 11, wherein directing implementation of a control action comprises directing at least one of (i) triggering an alarm or (ii) communicating an alert message.

19. The method of claim 11, further comprising:
directing, by the monitoring system based at least in part upon the determined posture, activation of one or more cameras and/or audio receivers to facilitate additional monitoring of the monitored subject.

20. A method comprising:
receiving, by a monitoring system comprising one or more computers, data collected by one or more wave sensors configured to output waves, the data comprising information associated with respective time delays from the one or more wave sensors to a monitored subject in the path of the one or more wave sensors;
evaluating, by the monitoring system, the received data to determine a position and orientation of the monitored subject; and
determining, by the monitoring system based at least in part upon the determined position and orientation, whether an alert action should be taken.
